(19) United States
(12) Patent Application Publication
(10) Pub. No.: US 2017/0348466 A1
(43) Pub. Date: Dec. 7, 2017

(54) LASER TEXTURING SURFACE PREPARATION FOR PARYLENE COATING ADHESION

(71) Applicant: Cardiac Pacemakers, Inc., St. Paul, MN (US)

(72) Inventor: Eoin Enright, Castleconnell (IE)

(21) Appl. No.: 15/602,645

(22) Filed: May 23, 2017

Related U.S. Application Data
(60) Provisional application No. 62/344,080, filed on Jun. 1, 2016.

(51) Int. Cl. A61L 31/10; C23C 16/46; A61L 31/04; A61L 31/02; A61N 1/05; C23C 16/48; A61N 1/375

(52) U.S. Cl. CPC A61L 31/10; C23C 16/483; C23C 16/46; A61N 1/375; A61L 31/022; A61N 1/05; A61L 31/048; A61L 2400/18; A61L 2420/02

(57) ABSTRACT

A process for coating parylene onto a metal surface, such as a medical device, that has been textured by a series of laser pulses. The laser pulses can be overlapping or rastered. The textured portion of the metal surface and parylene coating can form a strong mechanical interlock. The bond created by using the laser texturing process can result in a cohesive failure of the parylene and not an adhesive failure of the bonding.

### CLAIM OF PRIORITY

This application claims the benefit of priority under 35 U.S.C. §119(e) of U.S. Provisional Patent Application Ser. No. 62/344,080, filed on Jun. 1, 2016, which is herein incorporated by reference in its entirety.

### TECHNICAL FIELD

### BACKGROUND

Parylene is a common material used across multiple industries, such as medical, electronic and aerospace. It has excellent chemical and electrical properties and is biocompatible. The material is a conformal coating that is deposited using a room temperature vacuum chemical vapor deposition (CVD) process. This ensures that the coating forms a layer of uniform thickness around the part. While parylene has some attractive properties, it does not bond very well to surfaces

### SUMMARY

An example process for coating a metal surface with parylene can include subjecting at least a portion of the surface to a series of laser pulses, and then depositing a layer of parylene onto the surface. The metal surface can, for example, be titanium. In various examples, deposited parylene can create a mechanical bond with features created on the metal surface by the series of laser pulses.

After subjecting the portion to a series of laser pulses, the surface impinged by each pulse can have a trough area and a peak along the perimeter of the trough area. The troughs and peaks formed by the series of pulses can yield a textured portion of the surface, onto which the parylene can be deposited. In some examples, at least a portion of each peak can define an undercut in relation to a surface proximal to the peak. Some example processes can include subjecting a surface to a series of overlapping laser pulses, rastered laser pulses, or overlapping rastered laser pulses. In some examples, the laser process can create a roughness, i.e. a ridge height between the trough and the peak, of approximately 1-2 microns in height.

In some examples, depositing a layer of parylene can include chemical vapor deposition. The chemical vapor deposition can include, for example, vaporizing and heating a parylene dimer, whereby a gaseous parylene monomer is formed, and exposing the metal surface to the gaseous parylene monomer, thereby forming a deposit of parylene on a portion of the surface. In some example processes, the layer depth of parylene can be about 0.1 to about 200 microns, about 0.5 to about 80 microns, about 1 to about 30 microns, about 3 to about 25 microns, or about 5 to about 20 microns. In some examples, the parylene can be selected from the group the consisting of parylene N, parylene C, parylene D, parylene HT, parylene AF-4, parylene VT-4, parylene CF, and combinations thereof. The parylene can, for example, be parylene C.

Depositing a layer or parylene onto the surface can include depositing a layer of parylene to a depth of about 5 to about 20 microns onto the surface. The depositing can include vaporizing and heating a dimer of parylene C, whereby gaseous parylene C monomer is formed and exposing the metal surface to the gaseous parylene C monomer, thereby forming a deposit of parylene C on the portion of the surface. In some examples, the deposited parylene can extend under at least a portion of an undercut region formed on a metal surface. In various examples, deposited parylene can create a mechanical bond with features created by one or more laser pulses on the metal surface. For example, an undercut region can be formed by a laser pulse or series of laser pulses. In some examples, parylene extending under a portion of an undercut region can create a mechanical interlock with the metal surface. In some examples, a strength of a mechanical interlock created by the titanium-parylene coating process on a laser textured surface is stronger than the tensile strength of the parylene coating.

An example medical device can include at least one metal surface, a portion of which is textured by a plurality of troughs and peaks, each trough defining an area having a peak along the perimeter of the trough area, and at least a portion of each peak defining an undercut in relation to the surface proximal to the peak. In some examples, the laser process can create a roughness, i.e. a ridge height between the trough and the peak, of approximately 1-2 microns in height. The medical device can also include a layer of parylene on the metal surface. In some examples, the parylene can be one of the group the consisting of parylene N, parylene C, parylene D, parylene HT, parylene AF-4, parylene VT-4, parylene CF, and combinations thereof In an example, the parylene is parylene C. In various examples, the layer depth of parylene is about 0.1 to about 200 microns, about 0.5 to about 80 microns, about 1 to about 30 microns, about 3 to about 25 microns, about 5 to about 20 microns, or about 10 to 15 microns. In various examples, deposited parylene can create a mechanical bond with features created by one or more laser pulses on the metal surface. In some examples, deposited parylene can extend under at least a portion of an undercut region formed on a metal surface, which can create a mechanical interlock between the parylene and the metal surface. In some examples, a strength of a mechanical interlock of the titanium-parylene coating on the laser textured surface is stronger than the tensile strength of the parylene coating.

An example process for coating a metal surface with parylene can include subjecting at least one portion of the metal surface to a series of laser pulses, wherein the surface impinged by each pulse has a trough area and a peak along the perimeter of the trough area, the series pulses thereby yielding a textured portion of the metal surface. The metal can for example, be titanium. In some examples, the laser process can create a roughness, i.e. a ridge height between the trough and the peak, of approximately 1-2 microns in height. The process can further include depositing a layer of parylene onto the surface. In some examples, the process can include subjecting a portion of the metal surface to a series of overlapping laser pulses, rastered laser pulses, or overlapping rastered laser pulses. In some examples, the laser pulses can create an undercut in relation to the surface proximal to the peak. In some examples, depositing a layer of parylene can include chemical vapor deposition. The chemical vapor deposition can include vaporizing and heating a parylene dimer, whereby a gaseous parylene monomer is formed, and exposing the metal surface to the gaseous parylene monomer, thereby forming a deposit of parylene on a portion of the surface. The parylene can, for example, be selected from the group the consisting of parylene N, parylene C, parylene D, parylene HT, parylene AF-4, parylene VT-4, parylene CF, and combinations thereof In an example, the parylene is parylene C. In various examples, deposited parylene can create a mechanical bond with features created by one or more laser pulses on the metal surface. In some examples, parylene extending under a portion of an undercut region can create a mechanical interlock with the metal surface. In some examples, a strength of a mechanical interlock created by the titanium-parylene coating process on a laser textured surface is stronger than the tensile strength of the parylene coating.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a scanning electron microscope (SEM) image of the surface of a titanium coupon (plan view) that was textured using the laser process described herein.

FIG. 2 is an SEM image of the surface of a titanium coupon (oblique view) that was textured using the laser process described herein.

FIG. 3 is an SEM image of the surface of another titanium coupon that was textured using the laser process described herein.

FIG. 4 shows a titanium coupon with laser texturing applied to the left half, thereby allowing for a comparison of bonding of a parylene coating to both the native titanium surface (right half) and the laser textured surface (left half).

FIG. 5 shows a tested titanium coupon with a ˜0.025 mm parylene coating that has de-laminated from a non-textured half (right) to expose a native titanium surface in comparison to a strong and intact parylene bond to a textured half (left).

FIG. 6 shows a close-up view of the torn edges of a parylene-coated textured titanium surface, revealing strength of the titanium-parylene coating that is stronger than the tensile strength of the parylene coating.

FIG. 7A is a flow chart illustration of an example process.

FIG. 7B is a flow chart illustration of an example process.

FIG. 7C is a flow chart illustration of an example process.

### DETAILED DESCRIPTION

In the following detailed description, reference is made to the accompanying drawings which form a part hereof, and specific embodiments in which the disclosure may be practiced are shown by way of illustration. It is to be understood that other embodiments may be used and structural changes may be made without departing from the scope of the present disclosure.

While parylene has some attractive properties, it does not bond very well to surfaces and, hence, surface treatments can be implemented to ensure that the parylene bonds to a given surface. If there is insufficient bonding the parylene coating can delaminate from the surface causing potential failure of the application. One approach to a surface treatment can use a chemical adhesion promoter such as a silane. This is a wet chemical process that involves a number of cleaning, application and drying steps. Once dry, the silane forms a bond to the surface and allows parylene to then bond to the silane layer.

The silane adhesion promotion process has a number of drawbacks. The process requires a number of time consuming process steps. It also uses a number of chemicals and solvents which are both hazardous to the environment and to human health. The silane adhesion promotion process has a short window after application to complete the parylene coating process. Further, because the silane-coated surface does not remain chemically active indefinitely, the process must be repeated if a certain coating time is exceeded.

Titanium and other metals are useful materials in medical and aerospace applications due to a combination of their mechanical properties and biocompatibility. Titanium, for instance, is one of the most common materials used for enclosure of active medical electronics and components. A number of devices and components using titanium require a coating of parylene for functionality. This may be as a moisture barrier, to reduce friction, or as a dielectric layer. Yet, coating titanium with parylene suffers from the disadvantages mentioned above.

To allow for a strong mechanical interlock between the metal substrate and an applied parylene coating, a laser system can be used to create a texture on a surface of a metal substrate. Texturing metal surfaces with a laser process as described herein can provide a strong bond between the metal substrate and a parylene coating. For example, laser texturing of a metal surface process can create troughs and peaks at sites of laser pulse impingement. In some examples, a laser process creates undercut regions that extends under a peaks. The undercut region can engage with parylene to create a mechanical bond. By contrast, untextured (flat) surfaces can yield much weaker bonding characteristics with parylene. In some examples, texturing can alleviating the need for conventional enhancements, such as silane deposition, to promote a bond between parylene and substrate.

In examples wherein laser pulses sufficiently overlap with each other, the texturing process can provide an additional benefit of cleaning the surface due to the high temperatures of the laser pulse.

In some examples, the laser process can create a roughness, i.e. a ridge height between the trough and the peak, of approximately 1-2 microns in height. Because the texturing affects only the very top surface of the metal, the process does not disturb the structural integrity of the metal. In addition to the relatively low laser power required for the texturing, the process can use a high scan speed and thereby minimize heat build-up in the substrate.

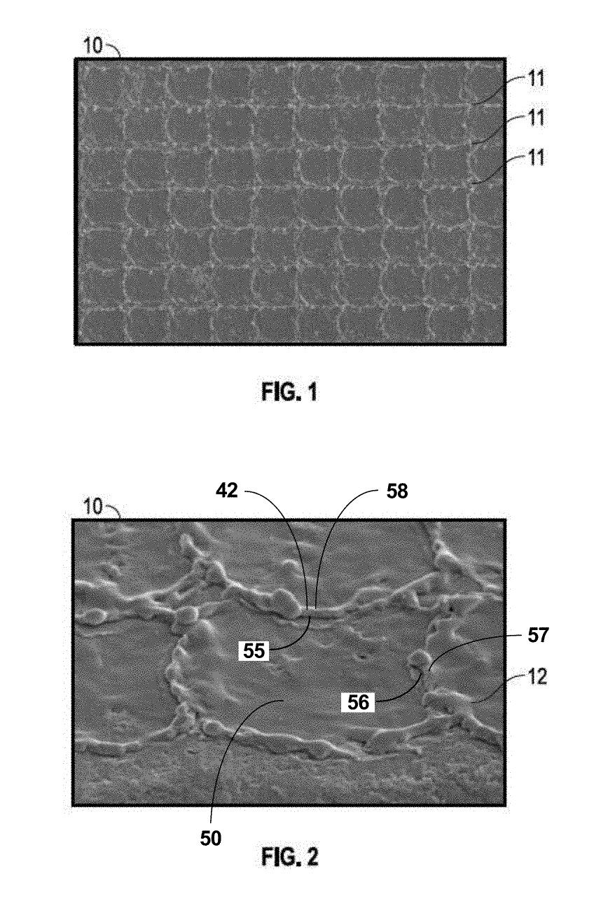

In some examples, rastered laser pulses can overlap with each other, as shown, for instance, in FIGS. 1 and 2. Alternatively, according to other examples, the laser pulses can be spaced so as to not overlap, either partially or completely. For instance, laser pulses overlapping along one dimension do not, in the aggregate, overlap with parallel lines of pulses. In another example, none of the laser pulses overlap with each other.

The laser texturing technique can be applicable to a variety of metallic substrates. The laser type, parameters, and optics can be adjusted for the differences in material type. For instance, some examples relate to medical devices, for which titanium is a typical metal. Commercially pure titanium grades 1-5 are suitable for this purpose, grade 2 being an exemplary grade of titanium.

In other examples, a useful metal is a titanium alloy. An example is Ti6Al4V (grade 5). Other examples include alpha alloys such as Ti-5AL-2SN-ELI and Ti-8AL-1MO-1V; near-alpha alloys such as Ti-6Al-2Sn-4Zr-2Mo, Ti-5Al-5Sn-2Zr-2Mo, IMI 685, and Ti 1100; alpha and beta alloys such as Ti-6Al-4V, Ti-6Al-4V-ELI, Ti-6Al-6V-2Sn, and beta and near-beta alloys such as Ti-10V-2Fe-3Al, Ti-13V-11Cr-3Al, Ti-8Mo-8V-2Fe-3Al, Beta C, Ti-15-3. Still other examples include Ti6Al7Nb, Ti5Al2.5Fe, Ti13Nb13Zr, and Ti12Mo6Zr2Fe.

In other examples, the metal is stainless steel. Examples include 304, 316L, and 405.

Other exemplary metals include the nickel-cobalt-chromium-molybdenum alloy MP35N, and the nickel titanium alloy nitinol.

Other examples include precious metals. Examples are platinum and palladium, either pure or alloyed with iridium, and gold.

In addition, the metal in some examples is an alloy of cobalt-chromium-molybdenum or cobalt-chromium. A typical cobalt-chromium-molybdenum alloy is F75.

As generally described above, parylene has some attractive properties when used on devices whose surfaces are pre-coated, such as with silanes, to promote adhesion of parylene. One advantage of the claimed invention is the elimination of any pre-coating, such that parylene can be coated directly onto a device surface. The term “parylene,” as used herein, refers to any one or combination of parylene compounds. That is to say, the parylene family includes several members. Parylene N, for example, is a nonchlorinated poly(paraxylylene) that has a low dissipation factor, high dielectric strength, and a dielectric constant that does not vary with the frequency of electrical current. Parylene N is useful for penetrating and coating into a device's small crevices and spaces.

Another typical parylene according to some examples is Parylene C, which is a chlorinated derivative of Parylene N. Parylene C provides a useful combination of electrical and physical properties, plus a low permeability to moisture, fluids, and corrosive gases. Its ability to provide pinhole-free conformal barriers makes it the coating of choice for many critical medical electronic assemblies.

Another example is Parylene HT, which is a polyfluorinated parylene. It has the lowest dielectric constant and dissipation factor of all the parylenes, as well as the highest continuous service temperature (350° C.). It also maintains its properties despite exposure to UV light.

Parylene coatings may be applied using known techniques. In general, parylene is deposited by chemical vapor deposition. For instance, in some examples, the technique is vapor-deposition polymerization that is performed in a vacuum chamber at room temperature. Parylene deposition occurs on the molecular level, with the coating growing one molecule at a time. This allows parylene to penetrate and coat small cracks, crevices, and openings, and protect even hidden surfaces in areas where other coating methods such as sprays and brushes cannot reach. Chemical vapor deposition also provides a uniform coating thickness, even on irregular surfaces.

Parylene is deposited as a vapor, so it surrounds the target surface and perfectly follows its contours, encapsulating it. Parylene coatings can be ultrathin and pinhole-free. The only raw material used in the coating process is a parylene dimer. When heated under vacuum, the dimer sublimates then cracks into a monomer vapor, which flows and spontaneously polymerizes onto all metal surfaces, forming an ultrathin, uniform film. No curing or additional steps are required.

In various examples, deposited parylene can create a mechanical bond with physical features created by one or more laser pulses on the metal surface. For example, the metal surface texturing process can create troughs and peaks at each site of laser pulse impingement, such that some or all of the peak portions of the ridge define an undercut structure. In some examples, a peak and undercut region can form a structure that is shaped analogous to a cresting wave frozen in time, and portions of the parylene can extend under the wave, i.e. into the undercut region.

Referring to the example shown in FIG. 2, a ridge (12) of material protrudes above an adjacent surface 50. An undercut region 56 extends beneath an overhang 57, which can interlock with parylene. Another undercut region 55 can be seen beneath overhang 58 on ridge 42. In some examples, the parylene layer indiscriminately penetrates various features of the textured surface, including the undercut portions, by which the parylene forms a three-dimensional interlocking layer with the textured surface.

The parylene layer in some embodiments measures about 0.1 to about 200 microns thick. Alternatively, the layer is about 0.5 to about 80 microns, about 1 to about 30 microns, about 3 to about 25 microns, or about 5 to about 20 microns thick. An exemplary range of layer thickness is about 5 to about 20 microns.

Additional non-limiting examples are set forth below.

### Additional Examples

### Methods and Materials

The substrate material that was used in these examples is commercially pure titanium grade 2 (CP—Ti). The material was stamped into a 16 mm diameter circle with a thickness of 0.3 mm. These coupon-sized samples provided sufficient area for experimentation, while having a sufficiently small overall size to allow laboratory analysis. The sample coupons were cleaned using isopropyl alcohol to remove any surface contaminants prior to experimentation.

Parylene coating was conducted in a Para-tech Lab Top 3000 parylene coating instrument. A standard program was used for a given quantity of dimer material. The material used was Parylene C manufactured by Galentis under the trade name of Galxyl C. Parylene thickness was verified using a Mitutoyo QuantuMike micrometer.

A glass slide was placed in the coating equipment during the sample coating. The parylene peeled off the glass slide very easily, and thus the coating thickness was measured directly.

The titanium substrate was textured using a Trumpf 3130 laser in a Trumpf 1000 enclosure. This laser system was a 1064 nm solid state laser. A spot size of approximately 45 microns was obtained from a F160 lens. Preliminary experiments were carried out to investigate the processing window, whereby a series of settings in the middle of this range were carried forward for this investigation.

The surfaces of the titanium coupons were placed at the focal distance of the lens. A laser scan speed of 3000 mm/sec with a laser pulse repetition rate of 80 kHz was used. The lasing pattern was a simple raster with a line to line spacing of 45 microns. Half of a coupon was textured to allow a direct comparison of the adhesion between the native titanium surface and textured surface.

The coated samples were mechanically damaged around the edge of the coupon to induce delamination of the parylene from the titanium surface. The coating was then lifted from the surface in a peeling motion to assess the bonding to the native and textured surface.

Optical images of the parts were taken using a Leica 205C stereo zoom microscope. High resolution images of the textured surface were taking using a Hitachi S-3600 scanning electron microscope.

Temperature measurements of the substrate were taken using a Pico TechnologyTC-08 thermocouple data logger attached to a PC.

### Results

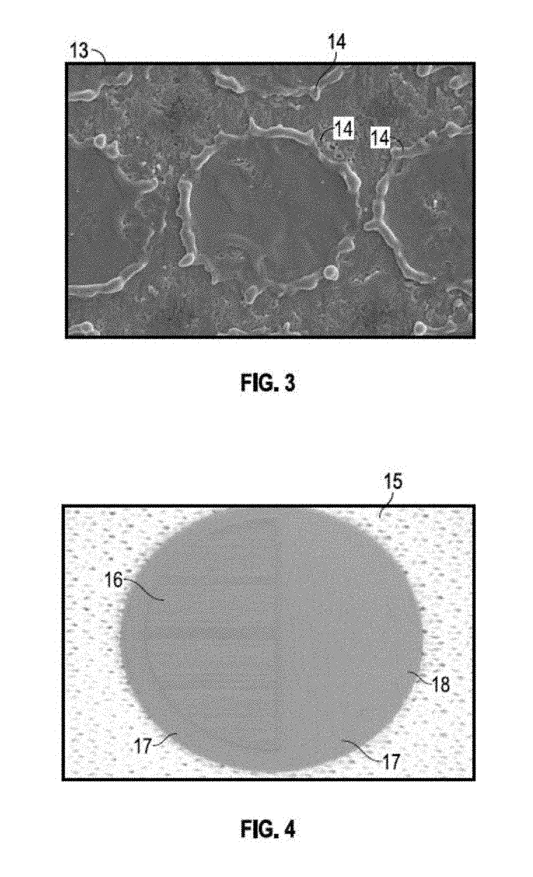

Texturing. Referring to FIG. 1, an SEM image (10) of the surface of the titanium that was textured using the laser process described above shows a slight overlapping pattern of the laser pulses to create a regular grid like pattern (11). As shown in a magnified view in FIG. 2, the laser pulse melted the surface of the titanium, creating a ridge of material (12) around its circumference. This ridge of material creates a three-dimensional undercut structure to which parylene can be adhered in a subsequent step. FIG. 2 shows ridge 12 with overhang 57 extending over undercut region 56, and ridge 42 with overhang 58 extending over undercut region 55. The density of these features per unit area can be altered by altering the gap between each laser pulse and the line to line spacing. FIG. 3 illustrates less dense spacing during the texturing process of a titanium surface (13) that resulted in non-overlapping ridges of material (14). This allows creation of an optimal surface structure for a given application.

In the example of FIG. 2, the SEM images show that the heights of the ridges of texture are approximately 1×10⁻⁶ m (1 micron). This height is approximately ˜0.3% in relation to the thickness of the tested substrate. In various examples, the height of the ridges can be 1-2 micron, and in some examples less than one micron. Because of this small size in comparison to typical medical device components, the ridge height can have little to no effect on the mechanical strength of the textured medical device component, which are typically at least one order of magnitude thicker than the ridge height. In addition, because the thickness of parylene coatings is generally several times greater than the ridge height, the height of the texture does not create a high point for wear. The low ridge height of the textured surface thus provides an additional advantage of not interfering with the performance of the parylene coating.

The laser pulses melting the top surface of the titanium material engendered localized temperatures exceeding 1650° C. As a consequence, the laser process can also be a cleaning process, such as removing any surface contamination, due to localized very high temperatures. The effect of temperature on the overall substrate temperature was monitored because excessive temperature could affect the substrate itself or any materials which may be in contact with the substrate. To monitor temperature, thermocouples were placed in direct contact with the back side of the coupon. Less than a 10° C. rise above ambient room temperatures (˜23° C.) was observed on the back side of the coupon, for the given laser settings and geometry.

Parylene Coating. Titanium coupons textured as described above were parylene coated. The parylene thickness was measured to be approximately 25 microns. This provided sufficient thickness in the film to assess the strength of the bonding to the surface. Thicker coatings allowed more transfer of force to the bond between the parylene and the titanium.

Testing Coated Surfaces. FIG. 4 illustrates a titanium coupon (15) that was textured only on its left half (16) as described above. The remainder of the coupon surface (17) was left untextured. The entire coupon (15) was then coated with parylene according to the procedure described above. The parylene was cut around the edge (18) of the coupon.

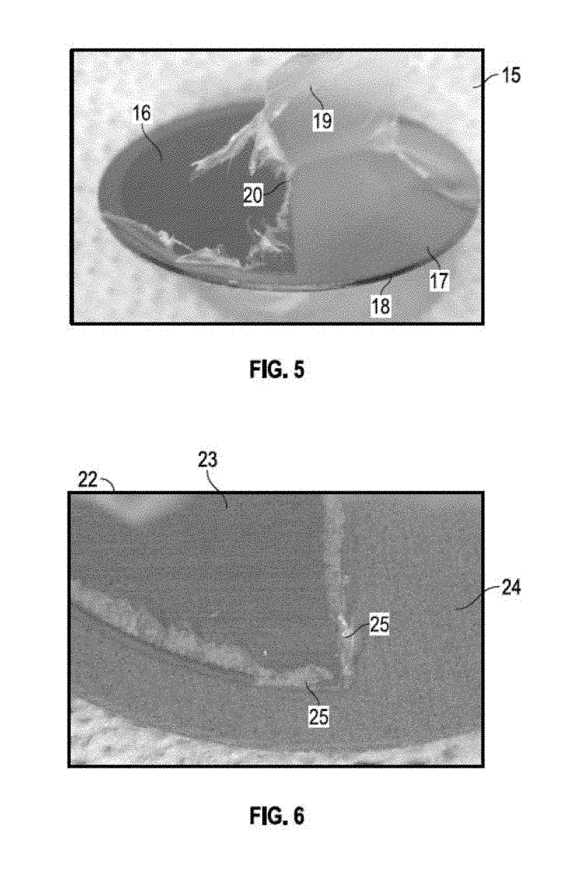

As shown in FIG. 5, the cut (18) allowed a section of parylene (19) to delaminate from the surface of the non-textured titanium (17). A tweezers was initially used to pull the parylene from the surface. The parylene delaminated from the native titanium surface (17) very easily.

By contrast, as shown in FIG. 5, parylene (19) did not delaminate from the laser textured area (16). A flap of delaminated parylene (19) was pulled by hand in a peeling manner at 90 degrees to the surface, to apply more force to the laser textured area (16). As a result, the parylene film was torn along the border (20) of the textured (16) and non-textured (17) surfaces. This indicates that the bond between the parylene and laser textured titanium was greater than the tensile strength of the parylene film for the given thickness.

FIG. 6 is a detailed image of an additional titanium coupon (22) that was prepared in a manner similar to that above, bearing a laser-textured left half (23) and non-textured remainder of the titanium surface (24), all coated with parylene in accordance with the procedure described above. Removal of the parylene coating revealed a tearing of the parylene coating at the boundary (25) between textured (23) and non-textured (24) portions of the titanium coupon surface, and evidencing high bond strength between parylene and the textured surface (23).

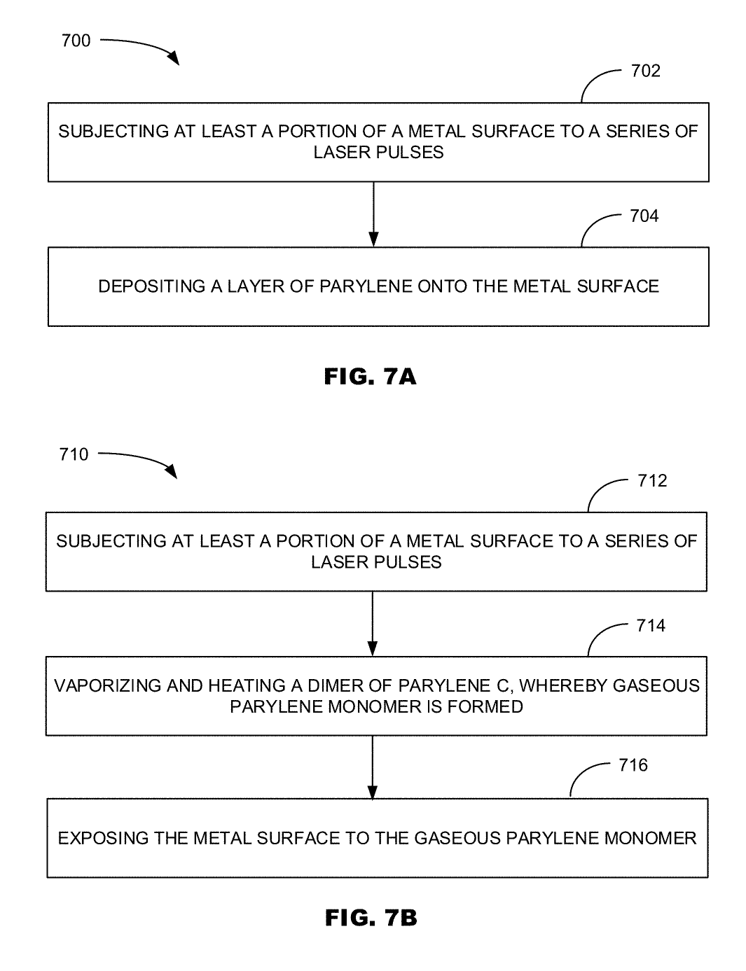

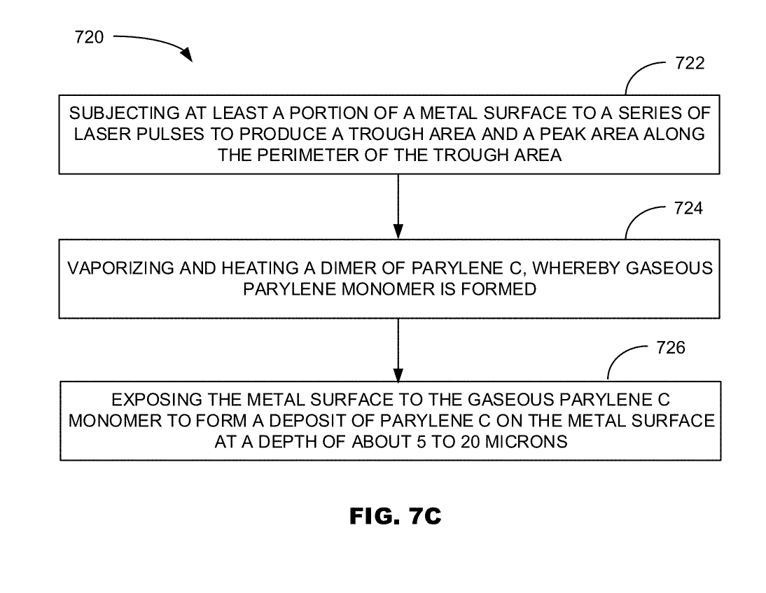

FIG. 7A is a flowchart illustration of an example process 700 for coating a metal surface with parylene. At step 702 a metal surface is subject to a series of laser pulses. The metal surface can be titanium, for example. In various examples, the laser pulses can be rastered laser pulses, overlapping laser pulses, or overlapping rastered laser pulses. The metal surface impinged by each pulse can have a trough area and a peak along the perimeter of the trough area. In an example, the trough and peak can provide a surface roughness (e.g., ridge height between trough and peak) of 1-2 microns. The series of pulses can yield a textured portion of the surface. In an example, after subjecting the metal surface to the laser pulses, at least a portion of each peak defines an undercut in relation to the surface proximal to the peak. In an example, undercut region has a shape analogous to a cresting wave. In an example, the metal surface can be a housing for an implantable medical device, such as a pacemaker, defibrillator, cardiac resynchronization therapy device, or neurostimulator, or an electrode for an implantable lead.

At step 704, a layer of parylene is deposited onto the metal surface. In an example, depositing the parylene onto the metal surface can include vaporizing and heating a parylene dimer, whereby a gaseous parylene monomer is formed, and exposing the metal surface to the gaseous parylene monomer, thereby forming a deposit of parylene on the portion of the surface. In an example, the parylene can be selected from the group the consisting of parylene N, parylene C, parylene D, parylene HT, parylene AF-4, parylene VT-4, parylene CF, and combinations thereof In an example, the parylene can be parylene C. In an example, the layer depth of parylene is deposited to depth of about 0.1 to about 200 microns, about 0.5 to about 80 microns, about 1 to about 30 microns, about 3 to about 25 microns, or about 5 to about 20 microns.

FIG. 7B is a flowchart illustration of an example process 710 for coating a metal surface with parylene. At step 712 a metal surface is subject to a series of laser pulses. In an example, the laser pulses can be rastered laser pulses. In an example, the laser pulses can be overlapping laser pulses. In an example, the laser pulses can be overlapping rastered laser pulses. The metal surface impinged by each pulse can have a trough area and a peak along the perimeter of the trough area. In an example, the trough and peak can provide a surface roughness (e.g., ridge height between trough and peak) of 1-2 microns. A series of pulses can yield a textured portion of the surface. In an example, after subjecting the metal surface to the laser pulses, at least a portion of each peak defines an undercut in relation to the surface proximal to the peak. In an example, the undercut region can be shaped like a cresting wave. In an example, the metal surface can be titanium. In an example, the metal surface can be a housing for an implantable medical device, such as a pacemaker, defibrillator, cardiac resynchronization therapy device, or neurostimulator.

Step 714 can include vaporizing and heating a parylene dimer, whereby a gaseous parylene monomer is formed. Step 716 can include exposing the metal surface to the gaseous parylene monomer, thereby forming a deposit of parylene on the portion of the surface. In an example, the parylene can be selected from the group the consisting of parylene N, parylene C, parylene D, parylene HT, parylene AF-4, parylene VT-4, parylene CF, and combinations thereof In an example, the parylene can be parylene C. In an example, the layer depth of parylene is deposited to depth of about 0.1 to about 200 microns, about 0.5 to about 80 microns, about 1 to about 30 microns, about 3 to about 25 microns, or about 5 to about 20 microns.

FIG. 7C is a flowchart illustration of an example process 720 for coating a metal surface with parylene. At step 722 a metal surface is subject to a series of laser pulses to produce a trough area and a peak area along the perimeter of the trough area. In an example, the trough and peak can provide a surface roughness (e.g., ridge height between trough and peak) of 1-2 microns. In an example, the laser pulses can be rastered laser pulses. In an example, the laser pulses can be overlapping laser pulses. In an example, the laser pulses can be overlapping rastered laser pulses. A series of pulses can yield a textured portion of the surface. In an example, after subjecting the metal surface to the laser pulses, at least a portion of each peak defines an undercut in relation to the surface proximal to the peak. In an example, the undercut region can be shaped like a cresting wave. In an example, the metal surface can be titanium. In an example, the metal surface can be a housing for an implantable medical device, such as a pacemaker, defibrillator, cardiac resynchronization therapy device, or neurostimulator.

Step 724 can include vaporizing and heating a parylene dimer, whereby a gaseous parylene monomer is formed. Step 726 can include exposing the metal surface to the gaseous parylene monomer, thereby forming a deposit of parylene on the portion of the surface at a depth of about 5 to 20 microns. In an example, the parylene can be selected from the group the consisting of parylene N, parylene C, parylene D, parylene HT, parylene AF-4, parylene VT-4, parylene CF, and combinations thereof In an example, the parylene can be parylene C.

A non-limiting numbered list of illustrative examples follows.

Example 1 can include or use subject matter (e.g., process, apparatus, article of manufacture, etc.) that can include or use a process for coating a metal surface with parylene. The process can include subjecting at least a portion of the metal surface to a series of laser pulses. The surface impinged by each pulse can have a trough area and a peak along the perimeter of the trough area. The series of pulses can yield a textured portion of the surface. A layer of parylene can be deposited onto the metal surface.

Example 2 can include or use, or can be combined with the subject matter of Example 1 to optionally include or use, depositing a layer of parylene onto the metal surface.

Example 3 can include or use, or can be combined with the subject matter of one or any combination of Examples 1-2, to include or use subjecting at least one portion of the surface to a series of overlapping laser pulses

Example 4 can include or use, or can be combined with the subject matter of one or any combination of Examples 1-3, to include or use subjecting the at least one portion of the surface to a series of rastered laser pulses.

Example 5 can include or use, or can be combined with the subject matter of one or any combination of Examples 1-4, to include or use depositing a layer of parylene including chemical vapor deposition. The chemical vapor deposition can comprise: vaporizing and heating a parylene dimer, whereby a gaseous parylene monomer can be formed; exposing the metal surface to the gaseous parylene monomer, thereby forming a deposit of parylene on the portion of the surface.

Example 6 can include or use, or can be combined with the subject matter of one or any combination of Examples 1-5, to include or use the parylene being selected from the group the consisting of parylene N, parylene C, parylene D, parylene HT, parylene AF-4, parylene VT-4, parylene CF, and combinations thereof.

Example 7 can include or use, or can be combined with the subject matter of one or any combination of Examples 1-6, to include or use the parylene being parylene C.

Example 8 can include or use, or can be combined with the subject matter of one or any combination of Examples 1-7, to include or use the layer depth of parylene being about 0.1 to about 200 microns, about 0.5 to about 80 microns, about 1 to about 30 microns, about 3 to about 25 microns, or about 5 to about 20 microns.

Example 9 can include or use, or can be combined with the subject matter of one or any combination of Examples 1-8, to include or use at least a portion of each peak defining an undercut in relation to the surface proximal to the peak.

Example 10 can include or use, or can be combined with the subject matter of one or any combination of Examples 1-9, to include or use the metal being titanium.

Example 11 can include or use, or can be combined with the subject matter of one or any combination of Examples 1-10 to include or use, after subjecting at least a portion of the surface to a series of laser pulses, at least a portion of each peak defining an undercut in relation to the surface proximal to the peak. Depositing a layer or parylene onto the surface can include depositing a layer of parylene to a depth of about 5 to about 20 microns onto the surface. The depositing can comprise: vaporizing and heating a dimer of parylene C, whereby gaseous parylene C monomer is formed; and exposing the metal surface to the gaseous parylene C monomer, thereby forming a deposit of parylene C on the portion of the surface.

Example 12 can include or use, or can be combined with the subject matter of one or any combination of Examples 1-11 to include or use a medical device comprising: at least one metal surface, a portion of which is textured by a plurality of troughs and peaks, each trough defining an area having a peak along the perimeter of the trough area; wherein at least a portion of each peak defines an undercut in relation to the surface proximal to the peak; and a layer of parylene on the metal surface.

Example 13 can include or use, or can be combined with the subject matter of one or any combination of Examples 1-12 to include or use the parylene being selected from the group the consisting of parylene N, parylene C, parylene D, parylene HT, parylene AF-4, parylene VT-4, parylene CF, and combinations thereof.

Example 13 can include or use, or can be combined with the subject matter of one or any combination of Examples 1-12 to include or use the parylene being parylene C.

Example 14 can include or use, or can be combined with the subject matter of one or any combination of Examples 1-13 to include or use the layer depth of parylene being about 0.1 to about 200 microns, about 0.5 to about 80 microns, about 1 to about 30 microns, about 3 to about 25 microns, or about 5 to about 20 microns.

Example 15 can include or use, or can be combined with the subject matter of one or any combination of Examples 1-14 to include or use the layer depth of parylene being about 5 to about 20 microns.

Example 16 can include or use, or can be combined with the subject matter of one or any combination of Examples 1-15 to include or use a process for coating a metal surface with parylene. The process can comprise: subjecting at least one portion of the surface to a series of laser pulses, wherein the surface impinged by each pulse has a trough area and a peak along the perimeter of the trough area, the series pulses thereby yielding a textured portion of the surface; and depositing a layer of parylene onto the surface.

Example 17 can include or use, or can be combined with the subject matter of one or any combination of Examples 1-16 to include or use subjecting the at least one portion to a series of overlapping laser pulses.

Example 18 can include or use, or can be combined with the subject matter of one or any combination of Examples 1-17 to include or use subjecting the at least one portion to a series of overlapping rastered laser pulses.

Example 19 can include or use, or can be combined with the subject matter of one or any combination of Examples 1-18 to include or use the depositing a layer of parylene includes chemical vapor deposition, the chemical vapor deposition comprising: vaporizing and heating a parylene dimer, whereby a gaseous parylene monomer is formed; exposing the metal surface to the gaseous parylene monomer, thereby forming a deposit of parylene on the portion of the surface.

Example 20 can include or use, or can be combined with the subject matter of one or any combination of Examples 1-19 to include or use the parylene being selected from the group the consisting of parylene N, parylene C, parylene D, parylene HT, parylene AF-4, parylene VT-4, parylene CF, and combinations thereof.

Example 21 can include or use, or can be combined with the subject matter of one or any combination of Examples 1-20 to include or use the parylene being parylene C.

Example 22 can include or use, or can be combined with the subject matter of one or any combination of Examples 1-21 to include or use the layer depth of parylene being about 0.1 to about 200 microns, about 0.5 to about 80 microns, or about 1 to about 30 microns.

Example 23 can include or use, or can be combined with the subject matter of one or any combination of Examples 1-21 to include or use the layer depth of parylene being about 3 to about 25 microns.

Example 24 can include or use, or can be combined with the subject matter of one or any combination of Examples 1-22 to include or use the layer depth of parylene being about 5 to about 20 microns.

Example 25 can include or use, or can be combined with the subject matter of one or any combination of Examples 1-24 to include or use at least a portion of each peak defining an undercut in relation to the surface proximal to the peak.

Example 25 can include or use, or can be combined with the subject matter of one or any combination of Examples 1-24 to include or use the metal being titanium.

Example 26 can include or use, or can be combined with the subject matter of one or any combination of Examples 1-25 to include or use a medical device. The medical device can comprise at least one metal surface, a portion of which is textured by a plurality of troughs and peaks, each trough defining an area having a peak along the perimeter of the trough area; wherein at least a portion of each peak defines an undercut in relation to the surface proximal to the peak; and a layer of parylene on the surface.

Example 27 can include or use, or can be combined with the subject matter of one or any combination of Examples 1-26 to include or use the parylene being selected from the group the consisting of parylene N, parylene C, parylene D, parylene HT, parylene AF-4, parylene VT-4, parylene CF, and combinations thereof.

Example 28 can include or use, or can be combined with the subject matter of one or any combination of Examples 1-26 to include or use the parylene being parylene C.

Example 29 can include or use, or can be combined with the subject matter of one or any combination of Examples 1-27 to include or use the layer depth of parylene being about 1 to about 30 microns.

Example 30 can include or use, or can be combined with the subject matter of one or any combination of Examples 1-29 to include or use the layer depth of parylene being about 3 to about 25 microns.

Example 31 can include or use, or can be combined with the subject matter of one or any combination of Examples 1-30 to include or use the layer depth of parylene being about 5 to about 20 microns.

Example 32 can include or use, or can be combined with the subject matter of one or any combination of Examples 1-31 to include or use the layer depth of parylene being about 10 to about 15 microns.

Example 33 can include or use, or can be combined with the subject matter of one or any combination of Examples 1-32 to include or use the metal being titanium.

Example 33 can include or use, or can be combined with the subject matter of one or any combination of Examples 1-32 to include or use the parylene being parylene C at a depth of about 5 to about 20 microns.

Example 35 can include or use, or can be combined with the subject matter of one or any combination of Examples 1-33 to include or use a process for coating a titanium surface with parylene C. The process can comprise: subjecting at least one portion of the surface to a series of laser pulses, wherein as a result of the laser pulses the surface incident to each pulse has a trough area and a peak along the perimeter of the trough area, and wherein at least a portion of each peak defines an undercut in relation to the surface proximal to the peak, the series of laser pulses thereby yielding a textured portion of the surface; and depositing a layer of parylene C to a depth of about 5 to about 20 microns onto the surface, the depositing can comprise: vaporizing and heating a dimer of parylene C, whereby gaseous parylene C monomer is formed; and exposing the metal surface to the gaseous parylene C monomer, thereby forming a deposit of parylene C on the portion of the surface.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

I claim:

1. A process for coating a metal surface with parylene, comprising:
subjecting at least one portion of the surface to a series of laser pulses,
wherein the surface impinged by each pulse has a trough area and a peak along the perimeter of the trough area,
the series pulses thereby yielding a textured portion of the surface; and
depositing a layer of parylene onto the surface.

2. The process according to claim 1, wherein the subjecting at least one portion of the surface to a series of laser pulses includes subjecting the at least one portion to a series of overlapping laser pulses.

3. The process according to claim 2, wherein the subjecting at least one portion of the surface to a series of laser pulses includes subjecting the at least one portion to a series of overlapping rastered laser pulses.

4. The process according to claim 1, wherein the depositing a layer of parylene includes chemical vapor deposition, the chemical vapor deposition comprising:
vaporizing and heating a parylene dimer, whereby a gaseous parylene monomer is formed;
exposing the metal surface to the gaseous parylene monomer, thereby forming a deposit of parylene on the portion of the surface.

5. The process according to claim 1, wherein the parylene is selected from the group the consisting of parylene N, parylene C, parylene D, parylene HT, parylene AF-4, parylene VT-4, parylene CF, and combinations thereof.

6. The process according to claim 5, wherein the parylene is parylene C.

7. The process according to claim 1, wherein the layer depth of parylene is about 0.1 to about 200 microns, about 0.5 to about 80 microns, or about 1 to about 30 microns.

8. The process according to claim 7, wherein the layer depth of parylene is about 3 to about 25 microns.

9. The process according to claim 8, wherein the layer depth of parylene is about 5 to about 20 microns.

10. The process according to claim 1, wherein at least a portion of each peak defines an undercut in relation to the surface proximal to the peak.

11. The process according to claim 1, wherein the metal is titanium.

12. A medical device comprising:
at least one metal surface, a portion of which is textured by a plurality of troughs and peaks, each trough defining an area having a peak along the perimeter of the trough area;
wherein at least a portion of each peak defines an undercut in relation to the surface proximal to the peak; and
a layer of parylene on the surface.

13. The medical device according to claim 12, wherein the parylene is selected from the group the consisting of parylene N, parylene C, parylene D, parylene HT, parylene AF-4, parylene VT-4, parylene CF, and combinations thereof

14. The medical device according to claim 13, wherein the parylene is parylene C.

15. The medical device according to claim 14, wherein the layer depth of parylene is about 1 to about 30 microns.

16. The medical device according to claim 15, wherein the layer depth of parylene is about 3 to about 25 microns.

17. The medical device according to claim 16, wherein the layer depth of parylene is about 5 to about 20 microns.

18. The medical device according to claim 17, wherein the layer depth of parylene is about 10 to about 15 microns.

19. The medical device according to claim 12, wherein
the metal is titanium; and
the parylene is parylene C at a depth of about 5 to about 20 microns.

20. A process for coating a titanium surface with parylene C, comprising:
subjecting at least one portion of the surface to a series of laser pulses,
wherein as a result of the laser pulses the surface incident to each pulse has a trough area and a peak along the perimeter of the trough area, and wherein at least a portion of each peak defines an undercut in relation to the surface proximal to the peak,
the series of laser pulses thereby yielding a textured portion of the surface; and
depositing a layer of parylene C to a depth of about 5 to about 20 microns onto the surface, the depositing comprising:
vaporizing and heating a dimer of parylene C, whereby gaseous parylene C monomer is formed; and
exposing the metal surface to the gaseous parylene C monomer, thereby forming a deposit of parylene C on the portion of the surface.
